(19) United States
(12) Patent Application Publication
(10) Pub. No.: US 2010/0087491 A1
(43) Pub. Date: Apr. 8, 2010

(54) POLYCYCLIC COMPOUNDS

(76) Inventors: Rainer Albert, Basel (CH); Nigel Graham Cooke, Oberwil (CH); Ian Lewis, Riehen (CH); Sven Weiler, Lorrach (DE); Frederic Zecri, Bartenheim (FR)

Correspondence Address: Fanelli Strain & Haag PLLC, 1455 Pennsylvania Ave., N.W., suite 400, Washington, DC, 20004, US

(21) Appl. No.: 12/443,233

(22) PCT Filed: Sep. 27, 2007
(86) PCT No.: PCT/EP07/08431

§ 371 Date: Mar. 27, 2009

(30) Foreign Application Priority Data
Sep. 29, 2006 (EP) 60121495.3

(51) Int. Cl. A61K 31/4439; C07D 271/06; C07D 413/04; A61K 31/4245; A61P 37/06

(52) U.S. Cl. 514/340; 548/131; 546/269.4; 514/364

(57) ABSTRACT

The invention relates to novel diaryl oxadiazole compounds of Formula I:

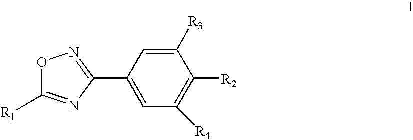

in which all the variables are as defined in the specification; to their preparation and to their use in the treatment of disorder or disease mediated by lymphocytes.

The present invention relates to polycyclic compounds, processes for their production, their use as pharmaceuticals and to pharmaceutical compositions comprising them.

More particularly the present invention provides in a first aspect a compound of formula I

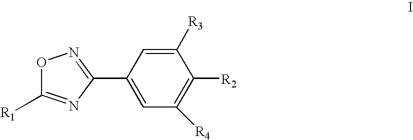

wherein
either
i. R₁ is a residue of formula (a)

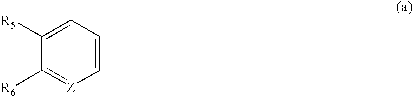

wherein Z is CH or N;
each of R₅ and R₆, independently, is H; halogen; C₁₋₈alkyl optionally substituted by OH, C₁₋₄alkoxy, ═N—OH or ═N—OC₁₋₄alkyl; halo-C₁₋₈alkyl; CN; C₁₋₈alkoxy; halo-C₁₋₈alkoxy; C₁₋₈alkylthio; Rₐ—CO, wherein Rₐ is C₁₋₄alkyl, C₃₋₆cycloalkyl, phenyl, or phenyl-C₁₋₄alkyl; or phenyl; or
R₅ and R₆ form together with the 2 carbon atoms to which they are attached another ring, for example a residue of formula (b),

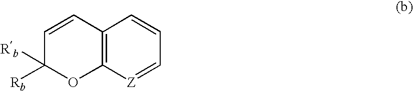

wherein Z is CH or N, preferably CH;
each of R_{b} and R′_{b}, independently, is C₁₋₄alkyl;
provided that at most one of R₅ and R₆ is H;
R₂ is —R₇—NR₈R₉ wherein R₇ is optionally substituted C₁₋₄alkylene; C₁₋₄alkylene wherein two bonds of a carbon atom of the alkyl chain form

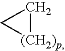

p being 1, 2 or 3; and
each of R₈ and R₉, independently, is H, optionally substituted C₁₋₈alkyl, R_{d}—CO or a heterocyclic group, or R₈ and R₉ form together with the nitrogen atom to which they are bound an optionally substituted heterocyclic group; and
R_{d} is C₁₋₄alkyl; C₃₋₆cycloalkyl; phenyl; or phenyl-C₁₋₄alkyl; or
R₂ is an optionally substituted heterocyclic group; and
each of R₃ and R₄ is H;
   provided that
1. when R₂ is CH₂—NH₂, then R₁ is other than 2-CF₃-4-biphenylyl, 3-CF₃-4-trifluoroethoxy-phenyl or 3-CF₃-4-isopropoxy-phenyl; and
2. when R₂ is C(CH₃)₂—NH₂, then R₁ is other than 2-CF₃-4-biphenylyl; or
ii) R₁ is substituted biphenylyl, 4-phenoxy-phenyl or 4-(phenyl-C₁₋₄alkoxy)-phenyl wherein at least one of the phenyl groups is monosubstituted; or phenyl substituted by one or more substituents selected from halogen, nitrile, C₁₋₈alkyl, haloC₁₋₈alkyl, C₁₋₈alkoxy, haloC₁₋₈alkoxy, C₁₋₈alkoxy-C₁₋₈alkoxy, C₁₋₈alkyl-C₁₋₈alkoxy, C₁₋₈alkyl-haloC₁₋₈alkoxy, haloC₁₋₈alkyl-C₁₋₈alkoxy, haloC₁₋₈alkyl-haloC₁₋₈alkoxy, haloC₁₋₈alkoxy-C₁₋₈alkoxy, C₁₋₈alkoxy-haloC₁₋₈alkoxy, haloC₁₋₈alkoxy-haloC₁₋₈alkoxy, C₁₋₈alkoxy-C₁₋₈alkyl, haloC₁₋₈alkoxy-C₁₋₈alkyl, C₁₋₈alkoxy-haloC₁₋₈alkyl, haloC₁₋₈alkoxy-haloC₁₋₈alkyl, C₂₋₆alkenyloxy, C₂₋₆alkynyloxy, C₃₋₆cycloalkyl, C₃₋₆cycloalkyl-C₁₋₄alkyl, C₃₋₆cycloalkyl-C₁₋₄alkoxy, C₃₋₆cycloalkyl-oxy, phenyl-C₁₋₄alkoxy and heterocyclic-C₁₋₄alkoxy;
   R₂ is SO₂NH₂; or SO₂NH—R₁₀—COOH wherein R₁₀ is C₁₋₆alkylene optionally interrupted by O, S or C═CH₂;
   one of R₃ and R₄ is H or C₁₋₄alkyl; and the other is C₁₋₄alkyl or haloC₁₋₄alkoxy;
   provided that when R₂ is SO₂NH₂ and R₄ is ethyl, then R₁ is other than
   a) biphenylyl substituted by CF₃ and optionally fluoro; or
   b) phenyl substituted by CF₃ and cyclohexyl;
      or a salt thereof.
In a compound of formula (I), the typical points of attachment in a residue of formula (a) are any of the positions being not occupied by a substituent R₅ or R₆. Preferably the substituents R₅ and R₆ are meta and para in relation to the oxadiazol ring.
By way of analogy, the typical points of attachment in a residue of formula (b) are any of the free positions in the ring carrying the group Z.

Halo or halogen may be fluorine, chlorine or bromine, preferably fluorine or chlorine. Alkyl or alkoxy as a group or present in a group may be straight or branched. C₁₋₄alkylene may be straight or branched.

Halo-alkyl or halo-alkoxy as a group or a moiety present in a group may be the corresponding alkyl or alkoxy group substituted by 1 to 5 halogen, e.g. CF₃ or CF₃—CH₂—O—.

Heterocyclic group may be a 5 or 6 membered saturated heterocyclic ring comprising 1 or 2 heteroatoms selected from N, S and O, and optionally substituted. Suitable examples include e.g. pyrrolidinyl, imidazolidinyl, pyrazolidinyl, piperidyl, piperazinyl or morpholino. When the heterocyclic ring is substituted, it may be a substituent on a cyclic carbon or nitrogen atom. Examples of a substituent on a cyclic carbon may be e.g. OH, C₁₋₄alkyl C₁₋₄alkoxy or hydroxy-C₁₋₄alkylene. Examples of a substituent on a nitrogen atom may be C₁₋₄alkyl.

When R₇ is optionally substituted C₁₋₆ alkylene, the alkylene may be substituted by OH, C₁₋₄alkoxy, hydroxy-C₁₋₄alkylene and/or C₁₋₄alkoxy-C₁₋₄alkylene.

When R₈ or R₉ is optionally substituted C₁₋₈alkyl, the substitutent may be OH or C₁₋₄alkoxy; preferably the substituent is on the terminal carbon atom of the alkyl chain.

When R₈ and R₉ form together with the nitrogen atom to which they are bound a heterocyclic group, it may be a heterocyclic ring as defined above, except that the heterocyclic residue formed by R₈ and R₉ is attached by a nitrogen atom.

When R₁ is substituted biphenylyl, 4-phenoxy-phenyl or 4-(phenyl-C₁₋₄alkoxy)-phenyl, either one and/or both phenyl moieties may be substituted, e.g. mono- or di-substituted e.g. by halogen, C₁₋₈alkyl, C₁₋₈alkoxy, haloC₁₋₈alkyl, haloC₁₋₈alkoxy or nitrile. Preferably at least one phenyl moiety of the biphenylyl, 4-phenoxy-phenyl or 4-(phenyl-C₁₋₄alkoxy)-phenyl is monosubstituted, e.g. as indicated above. Alternatively each phenyl moiety of the biphenylyl, 4-phenoxy-phenyl or 4-(phenyl-C₁₋₄alkoxy)-phenyl is monosubstituted, e.g. as indicated above, e.g. by haloC₁₋₈alkyl, and optionally as substitutent on the second phenyl moiety either halogen, C₁₋₈alkyl or C₁₋₈alkoxy, haloC₁₋₈alkyl or haloC₁₋₈alkoxy.

When R₁ is substituted phenyl, it may be mono- or di-substituted. When R₁ is disubstituted phenyl, one substituent may preferably be haloC₁₋₈alkyl or haloC₁₋₈alkoxy and the second substitutent as indicated above.

For the compounds of formula I the following significances are preferred independently, collectively or in any combination or sub-combination:

(i) R₁ is a residue of formula (a);

(ii) Z is CH;

(iii) R₂ is R₇—NR₈R₉ or a heterocyclic ring attached by a carbon atom, wherein the variables have the meanings provided hereinbefore;

(iv) R₃ is H;

(v) R₅ and R₆ do not form another ring and are independently selected from H; halogen; C₁₋₈alkyl optionally substituted by OH, or C₁₋₄alkoxy; halo-C₁₋₈alkyl; CN; C₁₋₈alkoxy; halo-C₁₋₈alkoxy; and phenyl; provided that at least one of R₅ or R₆ is different from H;

(vi) R₅ and R₆ are other than H;

(vii) R₅ and R₆ are not both C₁₋₈alkoxy at the same time;

(viii) R₁ is a residue of formula (a) and R₂ is a heterocyclic ring attached at a carbon atom.

The compounds of formula I may exist in free form or in salt form, e.g. addition salts with e.g. organic or inorganic acids, for example, hydrochloric acid or acetic acid.

It will be appreciated that the compounds of formula I may exist in the form of optical isomers, racemates or diastereoisomers. For example, R₇ may comprise an asymmetric carbon atom when R₇ is branched alkylene or substituted alkylene. It is to be understood that the present invention embraces all enantiomers and conformers and their mixtures. Similar considerations apply in relation to starting materials exhibiting asymmetric carbon atoms as mentioned above.

The present invention also includes a process for the production of a compound of formula I, which process comprises

a) condensing a compound of formula II wherein R₁ is as defined above, or a functional derivative thereof, with a compound of formula III (route A); or

b) converting a compound of formula IV or V to a compound of formula I (route B or C), and recovering the resulting compound of formula I in free form or in form of a salt, and, where required converting the compound of formula I obtained in free form into the desired salt form or vice versa.

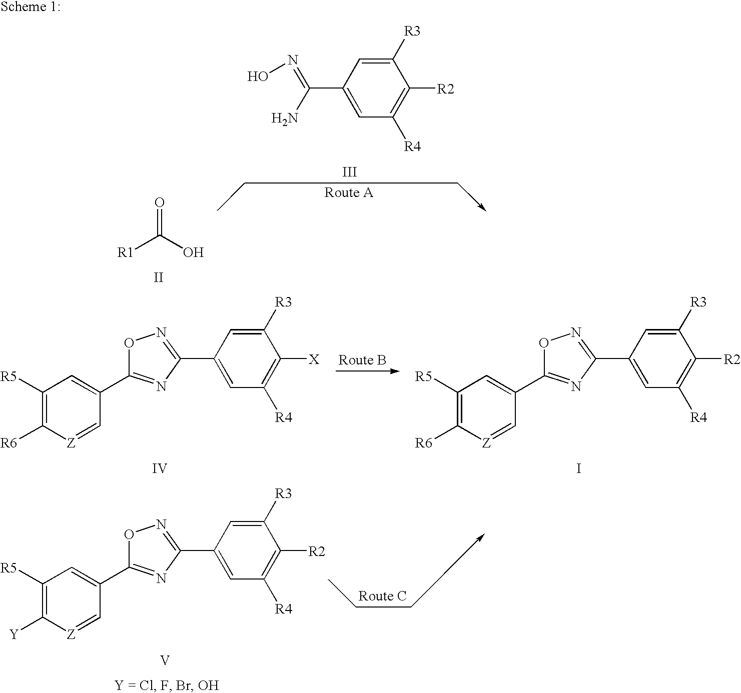

All reactions may be performed in a solvent e.g. methanol, ethanol, tetrahydrofuran, toluene, dichloromethane, 1,2-dichloroethane, N-methylpyrrolidone, xylenes, ethyl acetate, diethyl ether, hexanes, cyclohexanes, dimethylformamide, acetone, dimethylsulfoxide, tert-butylmethyl ether. All compounds may be isolated using methods known to those skilled in the art (e.g. crystallization, silica gel chromatography, HPLC).

Following route A, a compound of formula II may be condensed with a N-hydroxy amidine of formula III in the presence of a coupling agent, e.g. EDC or HOBt, and in the presence or absence of a suitable base (for example a tertiary amine such as Et₃N or Hünig's base) in a suitable solvent (for example dioxane, THF, toluene, DMF, acetonitrile). A functional derivative of an acid of formula II may be e.g. an acid chloride or an activated ester. The acid of formula II may be activated prior to the condensation as the corresponding acid chloride or as an activated ester (for example succinimide ester) (see scheme 2).

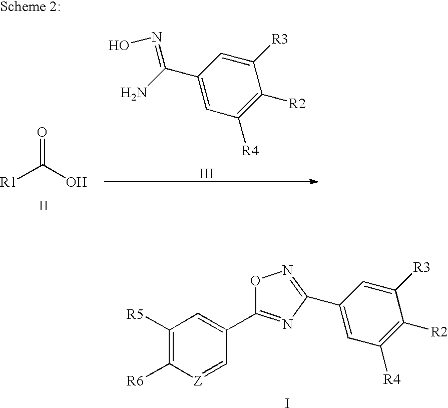

Following route B, compounds of formulae IV-a,b are converted respectively to compounds of formulae I-a,b by deprotection of either a carboxylic function or an amine function. Standard protecting group for carboxylic acid (for example esters) and for amine are used (for example carbamate). Using route B a compound of formula a to I-c is obtained by alkylation or acylation of the nitrogen atom of a compound of formula IV-c using methods known by in the art (see scheme 3).

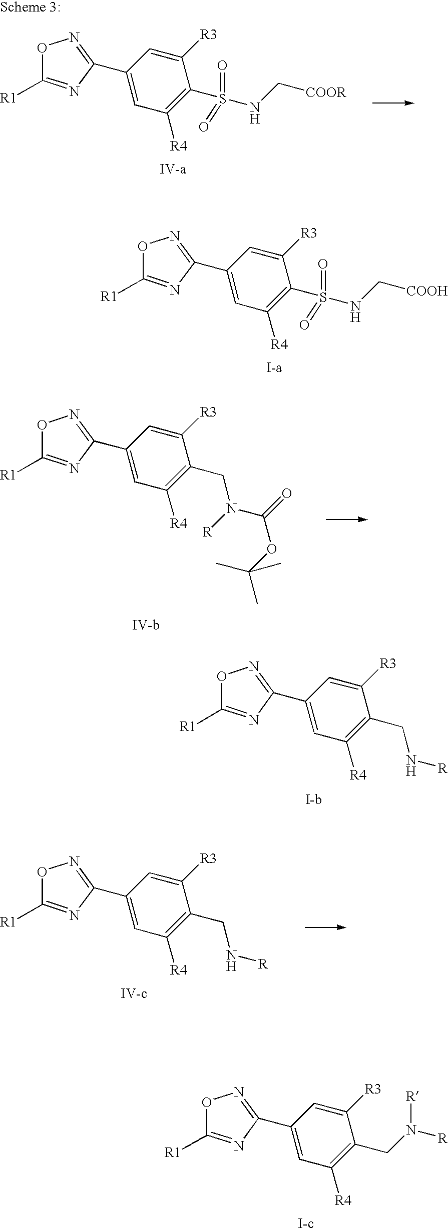

Following route C, a compounds of formula V may be converted into a compound of formula I wherein R₆ is C₁₋₄alkoxy by either alkylation of a compound of formula V wherein Y is OH, using standard conditions (e.g. using a base such as K₂CO₃, CsCO₃ or NaOH and a solvent, e.g. THF, ethanol, acetonitrile, acetone and the appropriate electrophilic alkylating agent) or by displacement of fluoride in a compound of formula V wherein Y is F, using standard conditions (for example using a base such as K₂CO₃, CsCO₃ or NaH and a solvent e.g. THF, acetonitrile, DMF and the appropriate alcohol) (see scheme 4).

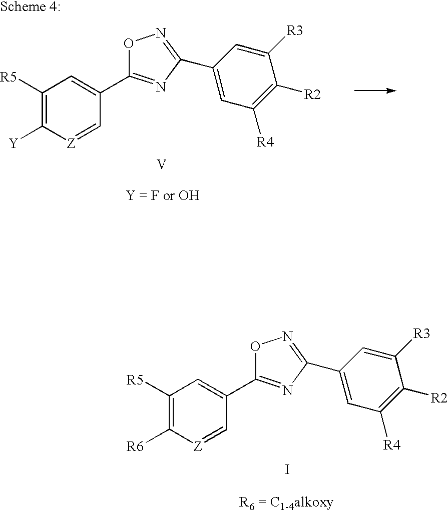

A compound of formula II, used as starting materials, may be prepared as follows:

Compounds of formulae VI-a,b if not commercially available or described in the literature may be synthesized following 2 routes. Biaryl carboxylic acids of formula VI-a may be obtained using Pd catalysed Suzuki conditions from 4-chloro benzoic acids and the corresponding aryl boronic acid (see scheme 5). 4-Alkoxy benzoic acids of formula VI-b may be obtained either by displacement of Y (when Y═F) using standard conditions (for example using a base such as K₂CO₃, CsCO₃ or NaH and a solvent such as THF, acetonitrile, DMF and the appropriate alcohol) or by alkylation (when Y is an hydroxy group) using standard conditions (for example using a base such as K₂CO₃, CsCO₃ or NaOH and a solvent such as THF, ethanol, acetonitrile, acetone and the appropriate electrophile) (see scheme 5)

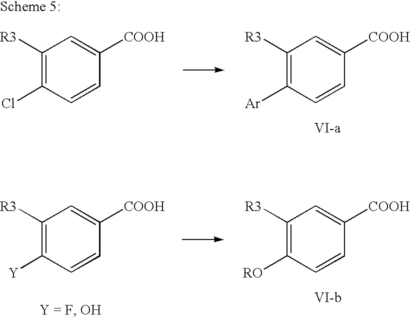

Compounds of formula III, used as starting materials, may be prepared as follows:

Compound of formula III if not commercially available or described in the literature may be prepared from the corresponding nitrile by condensation with hydroxylamine in a suitable solvent, e.g. water, ethanol, THF, dichloromethane (see scheme 6).

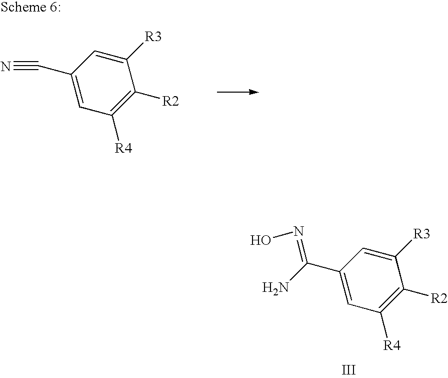

Insofar as the production of the starting materials is not particularly described, the compounds are either known or may be prepared analogously to methods known in the art or as disclosed hereinafter.

The following examples are illustrative of the invention, without any limitation. Concentration of solutions is carried out on a rotary evaporator under reduced pressure. Conventional flash chromatography is carried out on silica gel. Flash chromatography is also carried out using Biotage Flash Chromatography apparatus or Flashmaster instrument.

In a still further aspect the present invention relates to any aspect of the disclosed and described claims and/or examples individually, collectively or to any selections and/or any combinations thereof.

Abbreviations typically being used herein are:

TBME=tert.-butylmethyl ether

BOC=tert.-butyloxy carbonyl

Boc₂O=tert.-butyloxycarbonylanhydride

DMF=dimethylformamide

LiOH=lithium hydroxide

HCl=hydrochloric acid

THF=tetrahydrofuran

CH₂Cl₂=dichloromethane

RT=room temperature

NaOH=sodium hydroxide

HPLC=high pressure liquide chromatography

HOBt=hydroxybenzotriazole

EDC.HCl=1-ethyl-3-(3-dimethylaminopropyl)-carbodiimide hydrochloride

MS=mass spectroscopy

ES=electron spray

m/z=mass over charge number

### EXAMPLE 1

### 2-Trifluoromethoxy-4-[5-(2-trifluoromethyl-biphenyl-4-yl)-[1,2,4]oxadiazol-3-yl]-benzenesulfonamide

### a) 2-Trifluoromethyl-biphenyl-4-carboxylic acid

To a solution of 4-chloro-3-trifluoromethyl benzoic acid (15 g, 67 mmol) in THF (600 ml) is added under inert atmosphere phenylboronic acid (14.7 g, 120 mmol), dicyclohexylphosphino-2,4,6-triisopropylbiphenyl (3.18 g, 3.35 mmol), KF (11.65 g, 0.2 mol) and finally Pd(OAc)₂ (0.75 g, 6.7 mmol). The reaction mixture is then stirred under reflux for 1 hour. The reaction mixture is cooled and concentrated to dryness. Purification using flash chromatography afford the title compound.

m/z=265 (M−H)⁻

### b) 4-Bromo-2-trifluoromethoxy-benzenesulfonamide

To a solution of NH₄OH (25% in water) (3 ml, 44 mmol) in dioxane (10 ml) is added under inert atmosphere 4-bromo-2-trifluoromethoxy-benzenesulfonyl chloride (3 g, 8.8 mmol). The reaction mixture is then stirred at room temperature for 3 hours. The reaction mixture is concentrated to dryness, the residue is dried and recrystallized from diethylether/cyclohexane.

m/z=320 (M−H)⁻

### c) 4-Cyano-2-trifluoromethoxy-benzenesulfonamide

In a sealed tube, a solution of 4-bromo-2-trifluoromethoxy-benzenesulfonamide (2 g, 6.25 mmol) in N-methylpyrrolidone (8 ml) and CuCN (2 g, 22 mmol) is stirred and heated under microwave condition at 170° C. for 90 minutes. After cooling the reaction mixture is poured into water and extracted with methylenehloride. The organic layer is dried over Na₂SO₄, concentrated to dryness and purified on silica gel using cyclohexane/ethyl acetate 2/1→1/1 as mobile phase.

m/z=267 (M+H)⁺

### d) N-Hydroxy-4-sulfamoyl-3-trifluoromethoxy-benzamidine

To a solution of 4-cyano-2-trifluoromethoxy-benzenesulfonamide (1 g, 3.7 mmol) in THF (25 ml) is added at 5° C. a solution of 50% hydroxylamine in water (2.5 ml, 37.5 mmol). The reaction mixture is then stirred at room temperature for 16 hours. The reaction mixture diluted with water and THF is removed under vacuo. The resulting slurry is extracted with ethyl acetate, the organic phase is dried with Na₂SO₄ and concentrated. The residue is treated with diethylether to give the title compound as white precipitate.

m/z=300 (M+H)⁺

### e) 2-Trifluoromethoxy-4-[5-(2-trifluoromethyl-biphenyl-4-yl)-[1,2,4]oxadiazol-3-yl]-benzene-sulfonamide (Example 1)

To a solution of 2-trifluoromethyl-biphenyl-4-carboxylic acid (106 mg, 0.4 mmol) in DMF (2.5 ml) is added EDC.HCl (157 mg, 0.8 mmol) and HOBt (82 mg, 0.6 mmol), the reaction mixture is then stirred at room temperature for 30 minutes. Then N-hydroxy-4-sulfamoyl-3-trifluoromethoxy-benzamidine (120 mg, 0.4 mmol) is added to the reaction mixture and stirred for 30 minutes at room temperature, followed by stirring overnight at 95° C. The reaction mixture is then concentrated to dryness, dissolved in methylene chloride, extracted with 1N HCl followed by saturated NaHCO₃ solution and purified using flash chromatography to afford pure title compound.

m/z=528 (M−H)⁻

### EXAMPLE 6

### 4-{-4-[5-(2-Trifluoromethyl-biphenyl-4-yl)-[1,2,4]oxadiazol-3-yl]-benzyl}-morpholine

It is synthesized following the procedure of Example 1 replacing 4-cyano-2-trifluoromethoxy-benzenesulfonamide by 4-morpholin-4-ylmethyl-benzonitrile.

m/z=465.8 (M+H)⁺.

By following the procedure as described in Examples 1 or 6 and using methods described in Route A or B and using the appropriate starting materials the compounds of formula X₁

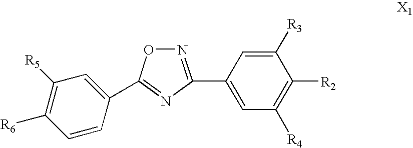

wherein R₅, R₆, R₂, R₃ and R₄ are as defined in Table 1 below, are obtained.

TABLE 1

| Ex | R₆ | R₅ | R₂ | R₃ | R₄ | ES-MS: |
|---|---|---|---|---|---|---|
| 1 | Phenyl | CF₃ | SO₂—NH₂ | O—CH₃ | H | m/z = 528 (M − H)⁻ |
| 2 | Phenyl | CF₃ | SO₂—NH₂ | CH₃ | CH₃ | m/z = 472 (M − H)⁻ |
| 3 | H | Phenyl | SO₂—NH₂ | CH₂—CH₃ | H | m/z = 404 (M − H)⁻ |
| 4 | F | Phenyl | SO₂—NH₂ | CH₂—CH₃ | H | m/z = 422 (M − H)⁻ |
| 5 | Methoxy | Phenyl | SO₂—NH₂ | CH₂—CH₃ | H | m/z = 434 (M − H)⁻ |
| 6 | Phenyl | CF₃ | CH₂—N- | H | H | m/z = 465.8 (M + H)⁺ |
| | | | morpholino | | | |

### EXAMPLE 7

### 3-(2-Ethyl-4-{5-[4-(2,2,2-trifluoro-ethoxy)-3-trifluoromethyl-phenyl]-[1,2,4]oxadiazol-3-yl}-benzenesulfonylamino)-propionic acid

### a) 3-(4-Bromo-2-ethyl-benzenesulfonylamino)-proplonic acid methyl ester

To a solution of 4-bromo-2-ethyl-benzenesulfonyl chloride (4.2 g, 14.8 mmol) in pyridine (20 ml) is added β-alanine methyl ester hydrochloride (4 g, 29.6 mmol). The reaction mixture is then stirred at room temperature for 16 hours. The reaction mixture is concentrated to dryness, the residue is dissolved in methylene chloride and extracted with 1N HCl. The organic layer is dried over Na₂SO₄, reduced and purified on silica gel using cyclohexane/ethyl acetate 2/1→1/1 as mobile phase.

m/z=351.5/353.5 (M+H)⁺.

### b) 3-(4-Cyano-2-ethyl-benzenesulfonylamino)-propionic acid methyl ester

A solution of 3-(4-bromo-2-ethyl-benzenesulfonylamino)-propionic acid methyl ester (1.2 g, 3.4 mmol) in N-methylpyrrolidone (3 ml) and CuCN (1.2 g, 13.4 mmol) is kept in a sealed tube at 170° C. (microwave) for 90 minutes. After cooling the reaction mixture is poured into water and extracted with methylene chloride. The organic layer is dried over Na₂SO₄, reduced and purified on silica gel using cyclohexane/ethyl acetate 2/1→1/1 as mobile phase.

m/z=294.9 (M−H)⁻.

### c) 3-[2-Ethyl-4-(N-hydroxycarbamimidoyl)-benzenesulfonylamino]-propionic acid methyl ester

To a solution of 3-(4-cyano-2-ethyl-benzenesulfonylamino)-propionic acid methyl ester (0.4 g, 1.4 mmol) in THF (10 ml) is added a 50% solution of hydroxylamine in water (1 ml, 14 mmol). The reaction mixture is stirred at room temperature for 48 hours. The reaction mixture is concentrated to dryness, then diluted with water and extracted with methylene chloride. The organic layer is dried over Na₂SO₄, reduced and purified on silica gel using cyclohexane/ethyl acetate 1/1→100% ethyl acetate as mobile phase.

m/z=329.8 (M+H)⁺.

### d) 3-{2-Ethyl-4-[5-(4-fluoro-3-trifluoromethyl-phenyl)-[1,2,4]oxadiazol-3-yl]-benzenesulfonyl-amino}-propionic acid methyl ester

To a solution of 4-fluoro-3-trifluoromethyl-benzoic acid (210 mg, 1 mmol) in DMF (15 ml) is added EDC.HCl (390 mg, 2 mmol) and HOBt (200 mg, 1.3 mmol), the reaction mixture is then stirred at room temperature for 30 minutes. Then 3-[2-ethyl-4-(N-hydroxycarbamimidoyl)-benzenesulfonylamino]-propionic acid methyl ester (225 mg, 0.7 mmol) is added to the reaction mixture and stirred for additional 30 minutes at room temperature, followed by stirring overnight at 95° C. The reaction mixture is then concentrated to dryness and purified using flash chromatography to afford 3-(2-Ethyl-4-{5-[4-(2,2,2-trifluoro-ethoxy)-3-trifluoromethyl-phenyl]-[1,2,4]oxadiazol-3-yl}-benzenesulfonyl-amino)-propionic acid methyl ester.

m/z=501.7 (M+H)⁺.

### e) 3-(2-Ethyl-4-{5-[4-(2,2,2,-trifluoro-ethoxy)-3-trifluoromethyl-phenyl]-[1,2,4]oxadiazol-3-yl}-benzenesulfonylamino)-proprionic acid

To a solution of 2,2,2-trifluoroethanol (110 mg, 1.1 mmol) in DMF (3 ml) is added NaH (60% in mineral oil) (21 mg, 0.53 mmol). After 10 minutes (clear solution) is added 3-{2-ethyl-4-[5-(4-fluoro-3-trifluoromethyl-phenyl)-[1,2,4]oxadiazol-3-yl]-benzenesulfonyl-amino}-propionic acid methyl ester (90 mg, 0.18 mmol) and the reaction mixture is stirred at room temperature for 2 hours. After removal of the solvent the residue is dissolved in methanol/water 1/1 (5 ml) and LiOH (20 mg, 0.83 mmol) is added. The reaction mixture is then stirred at 50° C. for 1 hour, then diluted with water, the pH is adjusted to ˜3 and the reaction is extracted with methylene chloride. The organic layer is dried over Na₂SO₄, evaporated and the residue is crystallized from cyclohexane or purified by flash chromatography.

ES-MS: m/z=566 (M−H)⁻.

### EXAMPLE 14

### 4-[5-(2-Methyl-biphenyl-4-yl)-[1,2,4]oxadiazol-3-yl]-benzylamine

### a) 2-Methyl-biphenyl-4-carboxylic acid

To a solution of 4-bromo-3-methyl-benzoic acid (2.5 g, 11.6 mmol) in THF (150 ml) is added under inert atmosphere phenylboronic acid (2.94 g, 23.4 mmol), dicyclohexylphosphino-2,4,6-triisopropylbiphenyl (570 mg, 1.16 mmol), KF (2.7 g, 60 mmol) and finally Pd(OAc)₂ (133 mg, 0.6 mmol). The reaction mixture is then stirred under reflux for 1 hour. The reaction mixture is cooled and concentrated to dryness. Purification using flash chromatography afford the title compound.

m/z=211 (M−H)⁻

### b) N-Boc-4-aminomethylbenzonitrile

To a solution of 4-aminoethylbenzonitrile hydrochloride (15 g, 94 mmol) in dioxane/1N NaOH 1/1 (450 ml) is added Boc₂O (28.8 g, 132 mmol). The reaction mixture is stirred at room temperature under inert atmosphere for 16 hours. The precipitate is collected and dried to yield N-boc-4-aminomethylbenzonitrile without further purification.

m/z=233.3 (M+H)⁺

### c) N-Boc-4-aminomethyl-N-hydroxy-benzamidine

To a solution of N-Boc-4-aminoethylbenzonitrile (14 g; 60 mmol) in THF (500 ml) is added at 5° C. a solution of hydroxylamine in water (1/1; 80 ml; 1.8 mol). The reaction mixture is then stirred at room temperature for 16 hours. The reaction mixture is diluted with water and THF is removed under vacuo. The resulting precipitate is collected by filtration and dried. Pure title compound is obtained by recrystallization from diethylether.

m/z=266.3 (M+H)⁺

### d) {4-[5-(2-Methyl-biphenyl-4-yl)-[1,2,4]oxadiazol-3-yl]-benzyl}-carbamic acid tert-butyl ester

To a solution of 2-methyl-biphenyl-4-carboxylic acid (234 mg; 1.1 mmol) in DMF (10 ml) is added EDC.HCl (394 mg; 2 mmol) and HOBt (205 mg; 1.5 mmol), the reaction mixture is then stirred at room temperature for 30 minutes. Then N-Boc-4-aminomethyl-N-hydroxy-benzamidine (266 mg; 1 mmol) is added to the reaction mixture and stirred for 30 minutes at room temperature, followed by stirring overnight at 95° C. The reaction mixture is then concentrated to dryness and purified using flash chromatography to afford the title compound.

m/z=442 (M+H)⁺

### e) 4-[5-(2-Methyl-biphenyl-4-yl)-[1,2,4]oxadiazol-3-yl]-benzylamine

A solution of {-4-[5-(2-methyl-biphenyl-4-yl)-[1,2,4]oxadiazol-3-yl]-benzyl}-carbamic acid tert-butyl ester (180 mg; 0.4 mmol) in TFA/H₂O 95/5 (1.8 ml; 19.5 mmol) is stirred at room temperature for 5 minutes. After addition of diethyl ether (25 ml) and HCl in diethyl ether (3M; 3 ml) the resulting precipitate is filtered off and dried.

m/z=341.9 (M+H)⁺

By following the procedure as described in above Examples and using methods described in Route A or B and using the appropriate starting materials the compounds of formula X₂

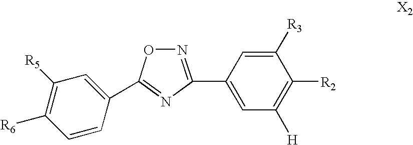

wherein R₅, R₆, R₂ and R₃ are as defined in Table 2 below, are obtained.

TABLE 2

| Ex | R₆ | R₅ | R₂ | R₃ | ES+/ES− MS: |
|---|---|---|---|---|---|
| 7 | 2,2,2-Trifluoro- | CF₃ | SO₂—NH—(CH₂)₂—COOH | CH₂—CH₃ | m/z = 566 (M − H)⁻ |
| | ethoxy | | | | |
| 8 | Isopropoxy | CF₃ | SO₂—NH—(CH₂)₂—COOH | CH₂—CH₃ | m/z = 526 (M − H)⁻ |
| 9 | Phenoxy | CF₃ | SO₂—NH—(CH₂)₂—COOH | CH₂—CH₃ | m/z = 544 (M − H)⁻ |
| 10 | 2,2,2-Trifluoro- | CF₃ | SO₂—NH—(CH₂)₂—COOH | CH₃ | m/z = 552 (M − H)⁻ |
| | ethoxy | | | | |
| 11 | Isopropoxy | CF₃ | SO₂—NH—(CH₂)₂—COOH | CH₃ | m/z = 512 (M − H)⁻ |
| 12 | Phenoxy | CF₃ | SO₂—NH—(CH₂)₂—COOH | CH₃ | m/z = 530 (M − H)⁻ |
| 13 | Phenyl | F | CH₂—NH₂ | H | m/z = 346.2 |
| | | | | | (M + H)⁺ |
| 14 | Phenyl | Methyl | CH₂—NH₂ | H | m/z = 341.9 |
| | | | | | (M + H)⁺ |
| 15 | Phenyl | Methoxy | CH₂—NH₂ | H | m/z = 358.0 |
| | | | | | (M + H)⁺ |
| 16 | Phenyl | Ethoxy | CH₂—NH₂ | H | m/z = 372.0 |
| | | | | | (M + H)⁺ |
| 17 | Phenyl | 2-Fluoro- | CH₂—NH₂ | H | m/z = 390.3 |
| | | ethoxy | | | (M + H)⁺ |
| 18 | Phenyl | 2,2,2-Tri- | CH₂—NH₂ | H | m/z = 426.0 |
| | | fluoro- | | | (M + H)⁺ |
| | | ethoxy | | | |
| 19 | 2- | CF₃ | CH₂—NH₂ | H | m/z = 381.9 |
| | Fluoroethoxy | | | | (M + H)⁺ |
| 20 | 2,2- | CF₃ | CH₂—NH₂ | H | m/z = 400.0 |
| | Difluoroethoxy | | | | (M + H)⁺ |
| 21 | Ethoxy- | CF₃ | CH₂—NH₂ | H | m/z = 363.9 |
| | | | | | (M + H)⁺ |
| 22 | n-Propoxy- | CF₃ | CH₂—NH₂ | H | m/z = 377.9 |
| | | | | | (M + H)⁺ |
| 23 | Ethoxy | Cl | CH₂—NH₂ | H | m/z = |
| | | | | | 330.7 (M + H)⁺ |
| 24 | Isopropoxy | Cl | CH₂—NH₂ | H | m/z = 344.0 |
| | | | | | (M + H)⁺ |
| 25 | Ethoxy | Br | CH₂—NH₂ | H | m/z = 373.8 |
| | | | | | (M + H)⁺ m/z = |
| | | | | | 375.8 (M + H)⁺ |
| 26 | Isopropoxy | Br | CH₂—NH₂ | H | m/z = 387.9 |
| | | | | | (M + H)⁺ m/z = |
| | | | | | 389.9 (M + H)⁺ |
| 27 | Ethoxy | CN | CH₂—NH₂ | H | m/z = 321.0 |
| | | | | | (M + H)⁺ |
| 28 | Isopropoxy | CN | CH₂—NH₂ | H | m/z = 335.0 |
| | | | | | (M + H)⁺ |
| 29 | 2,2,2-Trifluoro- | CN | CH₂—NH₂ | H | m/z = 375.0 |
| | ethoxy | | | | (M + H)⁺ |
| 30 | 2- | CN | CH₂—NH₂ | H | m/z = 339.0 |
| | Fluoroethoxy | | | | (M + H)⁺ |
| 31 | 2,2- | CN | CH₂—NH₂ | H | m/z = 357.0 |
| | Difluoroethoxy | | | | (M + H)⁺ |
| 32 | Ethoxy | Methyl | CH₂—NH₂ | H | m/z = 310.2 |
| | | | | | (M + H)⁺ |
| 33 | Isopropoxy | Methyl | CH₂—NH₂ | H | m/z = 324.3 |
| | | | | | (M + H)⁺ |
| 34 | Ethoxy | Methoxy | CH₂—NH₂ | H | m/z = 326.2 |
| | | | | | (M + H)⁺ |
| 35 | Methyl | Methoxy | CH₂—NH₂ | H | m/z = 296.1 |
| | | | | | (M + H)⁺ |
| 36 | Isopropoxy | Methoxy | CH₂—NH₂ | H | m/z = 340.3 |
| | | | | | (M + H)⁺ |
| 37 | Isobutoxy | Methoxy | CH₂—NH₂ | H | m/z = 354.3 |
| | | | | | (M + H)⁺ |
| 38 | Ethoxy | tert.-Butyl | CH₂—NH₂ | H | m/z = 352.1 |
| | | | | | (M + H)⁺ |
| 39 | Isopropoxy | tert.-Butyl | CH₂—NH₂ | H | m/z = 366.1 |
| | | | | | (M + H)⁺ |
| 40 | Ethoxy | Acetyl | CH₂—NH₂ | H | m/z = 338.0 |
| | | | | | (M + H)⁺ |
| 41 | Isopropoxy | Acetyl | CH₂—NH₂ | H | m/z = 352.3 |
| | | | | | (M + H)⁺ |
| 42 | Isopropoxy | 2- | CH₂—NH₂ | H | m/z = 354.3 |
| | | Hydroxy- | | | (M + H)⁺ |
| | | ethyl | | | |
| 43 | 6-(2,2-Dimethyl-2H)- | | CH₂—NH₂ | H | m/z = 334.0 |
| | chromenyl | | | | |
| 44 | Phenyl | CF₃ | CH₂—NH—CH₃ | H | m/z = 410.2 |
| | | | | | (M + H)⁺ |
| 45 | Phenyl | CF₃ | CH₂—N(CH₃)₂ | H | m/z = 424.2 |
| | | | | | (M + H)⁺ |
| 46 | Phenyl | CF₃ | CH₂—NH—CO—CH₃ | H | m/z = 438.1 |
| | | | | | (M + H)⁺ |
| 47 | Phenyl | CF₃ | CH₂—N(CH₃)—CO—CH₃ | H | m/z = 452.1 |
| | | | | | (M + H)⁺ |
| 48 | Phenyl | Methyl | CH₂—NH—CH₃ | H | m/z = 356.3 |
| | | | | | (M + H)⁺ |
| 49 | Phenyl | Methyl | CH₂—N(CH₃)₂ | H | m/z = 370.4 |
| | | | | | (M + H)⁺ |
| 50 | Phenyl | Methyl | CH₂—NH—COCH₃ | H | m/z = 384.4 |
| | | | | | (M + H)⁺ |
| 51 | Phenyl | Methoxy | CH₂—NH—CH₃ | H | m/z = 372.3 |
| | | | | | (M + H)⁺ |
| 52 | Phenyl | Methoxy | CH₂—N(CH₃)₂ | H | m/z = 386.4 |
| | | | | | (M + H)⁺ |
| 53 | 2,2,2-Trifluoro- | CF₃ | CH₂—NH—CH₃ | H | m/z = 431.9 |
| | ethoxy | | | | (M + H)⁺ |
| 54 | 2,2,2-Trifluoro- | CF₃ | CH₂—N(CH₃)₂ | H | m/z = 445.9 |
| | ethoxy | | | | (M + H)⁺ |
| 55 | 2,2,2-Trifluoro- | CF₃ | CH₂—NH—CO—CH₃ | H | m/z = 460.1 |
| | ethoxy | | | | (M + H)⁺ |
| 56 | Isopropoxy | CF₃ | CH₂—NH—CH₃ | H | m/z = 392.2 |
| | | | | | (M + H)⁺ |
| 57 | Isopropoxy | CF₃ | CH₂—N(CH₃)₂ | H | m/z = 405.9 |
| | | | | | (M + H)⁺ |
| 58 | Isopropoxy | CF₃ | CH₂—NH—CO—CH₃ | H | m/z = 420.1 |
| | | | | | (M + H)⁺ |
| 59 | 2- | CF₃ | CH₂—NH—CH₃ | H | m/z = 396.3 |
| | Fluoroethoxy | | | | (M + H)⁺ |
| 60 | 2- | CF₃ | CH₂—N(CH₃)₂ | H | m/z = 410.3 |
| | Fluoroethoxy | | | | (M + H)⁺ |
| 61 | 2- | CF₃ | CH₂—NH—CO—CH₃ | H | m/z = 424.2 |
| | Fluoroethoxy | | | | (M + H)⁺ |
| 62 | 2,2- | CF₃ | CH₂—NH—CH₃ | H | m/z = 413.9 |
| | Difluoroethoxy | | | | (M + H)⁺ |
| 63 | 2,2- | CF₃ | CH₂—N(CH₃)₂ | H | m/z = 428.0 |
| | Difluoroethoxy | | | | (M + H)⁺ |
| 64 | 2,2- | CF₃ | CH₂—NH—CO—CH₃ | H | m/z = 442.0 |
| | Difluoroethoxy | | | | (M + H)⁺ |
| 65 | Isopropoxy | Methyl | CH₂—NH—CH₃ | H | m/z = 338.3 |
| | | | | | (M + H)⁺ |
| 66 | Isopropoxy | Methyl | CH₂—N(CH₃)₂ | H | m/z = 352.3 |
| | | | | | (M + H)⁺ |
| 67 | Isopropoxy | Methyl | CH₂—NH—COCH₃ | H | m/z = 366.3 |
| | | | | | (M + H)⁺ |
| 68 | Isopropoxy | Methyl | CH₂—N(CH₃)—CH₂—CH₂—OH | H | m/z = 382.3 |
| | | | | | (M + H)⁺ |
| 69 | Isopropoxy | Acetyl | CH₂—NH—COCH₃ | H | m/z = 394.3 |
| | | | | | (M + H)⁺ |
| 70 | CN | Phenyl | CH₂—NH₂ | H | m/z = 353.3 |
| | | | | | (M + H)⁺ |
| 71 | Methoxy | Phenyl | CH₂—NH₂ | H | m/z = 358.3 |
| | | | | | (M + H)⁺ |
| 72 | Isopropoxy | Phenyl | CH₂—NH₂ | H | m/z = 386.0 |
| | | | | | (M + H)⁺ |
| 73 | 2- | Phenyl | CH₂—NH₂ | H | m/z = 390.3 |
| | Fluoroethoxy | | | | (M + H)⁺ |
| 74 | Ethoxy | Phenyl | CH₂—NH₂ | H | m/z = 372.3 |
| | | | | | (M + H)⁺ |
| 75 | n-Propoxy | Phenyl | CH₂—NH₂ | H | m/z = 386.4 |
| | | | | | (M + H)⁺ |
| 76 | Phenyl | CF₃ | (S)-CH₂—CH(NH₂)—CH₂OH | H | m/z = 440.3 |
| | | | | | (M + H)⁺ |
| 77 | Phenyl | CF₃ | (R)-CH₂—CH(NH₂)—CH₂OH | H | m/z = 440.3 |
| | | | | | (M + H)⁺ |
| 78 | Phenyl | CF₃ | (S)-CH₂—CH(NH₂)—COOH | H | m/z = 454.3 |
| | | | | | (M + H)⁺ |
| 79 | Phenyl | CF₃ | (R)-CH₂—CH(NH₂)—COOH | H | m/z = 454.3 |
| | | | | | (M + H)⁺ |
| 80 | Phenyl | Methoxy | (S)-CH₂—CH(NH₂)—CH₂OH | H | m/z = 402.3 |
| | | | | | (M + H)⁺ |
| 81 | Phenyl | Methoxy | (R)-CH₂—CH(NH₂)—CH₂OH | H | m/z = 402.3 |
| | | | | | (M + H)⁺ |
| 82 | Isopropoxy | Methyl | (S)-CH₂—CH(NH₂)—CH₂OH | H | m/z = 386.4 |
| | | | | | (M + H)⁺ |
| 83 | Isopropoxy | Methyl | 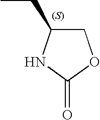 | H | m/z = 394.3 (M + H)⁺ |
| 84 | Phenyl | CF₃ | (R)-CH(NH₂)—CH₂—CH₂OH | H | m/z = 440.3 |
| | | | | | (M + H)⁺ |

### EXAMPLE 85

### 4-{5-[4-(2,2,2-Trifluoro-ethoxy)-3-trifluoromethyl-phenyl]-[1,2,4]oxadiazol-3-yl}-2-trifluoromethoxy-benzenesulfonamide

### a) 4-[5-(4-Fluoro-3-trifluoromethyl-phenyl)-[1,2,4]oxadiazol-3-yl]-2-trifluoromethoxy-benzene-sulfonamide

4-[5-(4-Fluoro-3-trifluoromethyl-phenyl)-[1,2,4]oxadiazol-3-yl]-2-trifluoromethoxy-benzene-sulfonamide is synthesized following the procedure of 2-trifluoromethoxy-4-[5-(2-trifluoromethyl-biphenyl-4-yl)-[1,2,4]oxadiazol-3-yl]-benzenesulfonamide (Example 1) replacing 2-trifluoromethyl-biphenyl-4-carboxylic acid by 4-fluoro-3-trifluoromethyl-benzoic acid.

m/z=470 (M−H)⁻

b) To a solution of 2,2,2-trifluoroethanol (63 mg, 0.63 mmol) in DMF (3 ml) is added NaH (60% in mineral oil) (20 mg, 0.5 mmol). The reaction mixture is then stirred for 10 minutes (clear solution), then is added 4-[5-(4-fluoro-3-trifluoromethyl-phenyl)-[1,2,4]oxadiazol-3-yl]-2-trifluoromethoxy-benzene-sulfonamide (118 mg, 0.25 mmol) and the reaction mixture is stirred at temperature for 16 hours. After removal of the solvent the product is obtained after silica gel chromatography using cyclohexane/ethyl acetate 4/1→2/1 as mobile phase.

m/z=550 (M−H)⁻

By following the procedure as described in above Examples and using methods described in Route A or B and using the appropriate starting materials the compounds of formula X₃

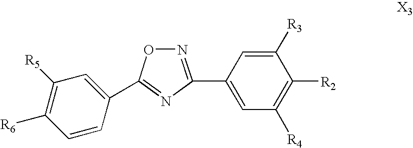

wherein R₅, R₆, R₂, R₃ and R₄ are as defined in Table 3 below, are obtained.

TABLE 3

| Ex | R₆ | R₅ | R₂ | R₃ | R₄ | ES-MS: |
|---|---|---|---|---|---|---|
| 85 | 2,2,2-Trifluoro-ethoxy | CF₃ | SO₂—NH₂ | O—CH₃ | H | m/z = 550 (M − H)⁻ |
| 86 | Isopropoxy | CF₃ | SO₂—NH₂ | O—CH₃ | H | m/z = 510 (M − H)⁻ |
| 87 | 2,2,2-Trifluoro-ethoxy | CF₃ | SO₂—NH₂ | CH₂—CH₃ | H | m/z = 494 (M − H)⁻ |
| 88 | Isopropoxy | CF₃ | SO₂—NH₂ | CH₂—CH₃ | H | m/z = 454 (M + H)⁺ |
| 89 | 2-Fluoro-phenoxy | CF₃ | SO₂—NH₂ | CH₂—CH₃ | H | m/z = 506 (M − H)⁻ |
| 90 | Phenoxy | CF₃ | SO₂—NH₂ | CH₂—CH₃ | H | m/z = 490 (M + H)⁺ |
| 91 | Cyclobutoxy | CF₃ | SO₂—NH₂ | CH₂—CH₃ | H | m/z = 468.1 (M⁺H)⁺ |
| 92 | 1-Methyl-2,2,2-trifluoro- | CF₃ | SO₂—NH₂ | CH₂—CH₃ | H | m/z = 508 (M − H)⁻ |
| | ethoxy | | | | | |
| 93 | 1-Methyl-2,2,2-trifluoro- | CF₃ | SO₂—NH₂ | CH₂—CH₃ | H | m/z = 508 (M − H)⁻ |
| | ethoxy | | | | | |
| 94 | 2,2,2-Trifluoro-ethoxy | CF₃ | SO₂—NH₂ | CH₃ | H | m/z = 480 (M − H)⁻ |
| 95 | Isopropoxy | CF₃ | SO₂—NH₂ | CH₃ | H | m/z = 440 (M − H)⁻ |
| 96 | 2-Fluoro-phenoxy | CF₃ | SO₂—NH₂ | CH₃ | H | m/z = 492 (M − H)⁻ |
| 97 | Phenoxy | CF₃ | SO₂—NH₂ | CH₃ | H | m/z = 476 (M + H)⁺ |
| 98 | 2,2,2-Trifluoro-ethoxy | CF₃ | SO₂—NH₂ | CH₃ | CH₃ | m/z = 494 (M − H)⁻ |
| 99 | Isopropoxy | CF₃ | SO₂—NH₂ | CH₃ | CH₃ | m/z = 454 (M − H)⁻ |
| 100 | 2-Fluoro-phenoxy | CF₃ | SO₂—NH₂ | CH₃ | CH₃ | m/z = 506 (M − H)⁻ |
| 101 | Phenoxy | CF₃ | SO₂—NH₂ | CH₃ | CH₃ | m/z = 488 (M − H)⁻ |
| 102 | 1-Methyl-2,2,2-trifluoro- | CF₃ | SO₂—NH₂ | CH₃ | CH₃ | m/z = 510.3 (M − H)⁻ |
| | ethoxy | | | | | |
| 103 | 1-Methyl-2,2,2-trifluoro- | CF₃ | SO₂—NH₂ | CH₃ | CH₃ | m/z = 510.3 (M − H)⁻ |
| | ethoxy | | | | | |
| 104 | 2,2,2-Trifluoro-ethoxy | Phenyl | SO₂—NH₂ | CH₂—CH₃ | H | m/z = 502 (M − H)⁻ |
| 105 | Isopropoxy | Phenyl | SO₂—NH₂ | CH₂—CH₃ | H | m/z = 464 (M + H)⁺ |
| 106 | Methoxy | Phenyl | SO₂—NH₂ | CH₂—CH₃ | H | m/z = 434 (M − H)⁻ |
| 107 | 2,2,2-Trifluoro-ethoxy | CF₃ | CH₂—N- | H | H | m/z = 488.6 (M + H)⁺ |
| | | | morpholino | | | |
| 108 | Isopropoxy | CF₃ | CH₂—N- | H | H | m/z = 448.6 (M + H)⁺ |
| | | | morpholino | | | |

The compounds of formula I in free form or in pharmaceutically acceptable salt form, exhibit valuable pharmacological properties, e.g. as S1P1 receptor agonists, e.g. as indicated in vitro and in vivo tests and are therefore indicated for therapy.

A. In vitro

The compounds of formula I have binding affinity to individual human S1P receptors as determined in following assays:

A. In Vitro: GPCR Activation Assay Measuring GTP [γ-³⁵S] Binding to Membranes Prepared from CHO Cells Expressing Human S1P Receptors

S1P, (EDG-1) GTP [γ-³⁵S] binding assay: Homogenized membranes are prepared from CHO cell clones stably expressing a human EDG-1 N-terminal c-myc tag. Cells are grown in suspension in two 850 cm² roller bottles for three or fours days before harvesting. The cells are centrifuged down, washed once with cold PBS, and resuspended in ≦20 ml of Buffer A (20 mM HEPES, pH 7.4, 0 mM EDTA, EDTA-free complete protease inhibitor cocktail [1 tablet/25 ml]). The cell suspension is homogenized on ice, using a Polytron homogenizer at 30000 rpm at three intervals of 15 seconds each. The homogenate is first centrifuged at 2000 rpm on a tabletop low speed centrifuge for 10 minutes. The supernatant, after passing through a cell strainer, is then re-centrifuged at 50,000×g for 25 minutes at 4° C. The pellet is resuspended into buffer B (15% glycerol, 20 mM HEPES, pH 7.4, 0.1 mM EDTA, EDTA-free complete protease inhibitor cocktail [1 tablet/10 ml]). Protein concentration of the preparation is determined using the BCA Protein Assay kit (Pierce) using BSA as standard. The membranes are aliquoted and kept frozen at −80° C.

Solutions of test compounds ranging from 10 mM to 0.01 nM are prepared in DMSO. S1P is diluted in 4% BSA solution as positive controls. The desired amount of membrane preparation is diluted with ice-cold assay buffer (20 mM HEPES, pH 7.4, 100 mM NaCl, 10 mM MgCl₂, 0.1% Fatty acid-free BSA, 5 μM GDP) and vortexed well. 2 μl or less of compound is distributed into each well of a round-bottom 96-well polystyrene assay plate, followed by addition of 100 μl of diluted membranes (3-10 μg/well) and kept on ice until the addition of hot GTPγS. [³⁵S]-GTPγS is diluted 1:1000 (v/v) with cold assay buffer and 100 μl is added into each well. The reaction is carried out at room temperature for 90 minutes before the membranes are harvested onto Perkin-Elmer Unifilter® GF/B-96 filter plate using a Packard Filtermate Harvester. After several washes with wash buffer (20 mM HEPES, pH 7.4, 100 mM NaCl, 10 mM MgCl₂), and a rinse with 95% ethanol, the filter is dried in a 37° C. oven for 30 minutes. MicroScint-20 is added and the plate sealed for scintillation counting on TopCount. EC₅₀ values are obtained by fitting the GTP [γ-³⁵S] binding curves (raw data) with the dose response curve-fitting tool of GraphPad Prism. Six or twelve different concentrations are used to generate a concentration response curve (using three data points per concentration).

S1P-2,-3,-4 and -5 GTP [γ-³⁵S] binding assays are carried out in a comparable manner to the S1P1 GTP [γ-³⁵S] binding assay using membranes from CHO cells stably expressing c-terminal c-myc tagged or untagged receptors. For each membrane preparation, titration experiments are first run with S1P control to determine the optimal amount of membranes to be added per assay well.

Compounds of formula I are tested according to the above assay and show binding affinity to to SIP receptors, e.g. S1P1 receptors with an EC₅₀1 μM. More particularly, compounds of formula I exhibit selectivity for the S1P1 receptor. For example, Compounds of Examples 21, 44 and 105 have an EC₅₀<100 nM in the above S1P1 receptor binding assay and are at least 20 fold selective for S1P1 receptor compared to S1P3 receptor, and at least 20 fold selective for S1P1 receptor compared to S1P-5 receptor.

### B. In Vitro: FLIPR Calcium Flux Assay

Compounds of formula I are tested for agonist activity on S1P1, S1P3, S1P5, and S1P6 with a FLIPR calcium flux assay. Briefly, CHO cells expressing an S1P receptor are maintained in F-12K medium (ATCC), containing 5% FBS, with 500 μg/ml of G418. Prior to the assay, the cells are plated in 384 black clear bottom plates at the density of 10,000 cells/well/25 μl in the medium of F-12K containing 1% FBS. The second day, the cells are washed three times (25 μl/each) with washing buffer. About 25 μl of dye are added to each well and incubated for 1 hour at 37° C. and 5% CO₂. The cells are then washed four times with washing buffer (25 μl/each). The calcium flux is assayed after adding 25 μl of SEW2871 (published by Rosen et al., used as reference) solution to each well of cells. The same assay is performed with cells expressing each of the different SIP receptors. Titration in the FLIPR calcium flux assay is recorded over a 3-minute interval, and quantitated as maximal peak height percentage response relative to S1P-1 activation. The compounds of formula I are active in this assay at a concentration of from 10⁻¹² and 3.10⁻⁵ M.

### C. In Vivo: Screening Assays for Measurement of Blood Lymphocyte Depletion

Measurement of circulating lymphocytes: Compounds to be tested are dissolved in DMSO/PEG200 and further diluted with deionized water. Rats (Lewis strain, female, 6-12 weeks old) are administered 1 mg/kg of compound to be tested in 4 ml/kg vehicle (max. 2% DMSO/max. 2% PEG200/water) via per os application. DMSO/PEG200/water and FTY720 (0.3 mg/kg) are included as negative and positive controls, respectively.

Blood is collected from the sublingual vein 2, 6, 24 and 48 hours after administration under short isoflurane anesthesia. Whole blood samples are subjected to hematology analysis. Peripheral lymphocyte counts are determined using an automated analyzer. Subpopulations of peripheral blood lymphocytes are stained by fluorochrome-conjugated specific antibodies and analyzed using a fluorescent activating cell sorter (Facscalibur). Two rats are used to assess the lymphocyte depletion activity of each compound screened. The result is an ED₅₀, which is defined as the effective dose required to display 50% of blood lymphocyte depletion. Compounds of formula I are tested according to the above assay and have an ED₅₀ of less than 10 mg/kg.

The compounds of formula I are, therefore, useful in the treatment and/or prevention of diseases or disorders mediated by lymphocytes interactions, e.g. in transplantation, such as acute or chronic rejection of cell, tissue or organ allo- or xenografts or delayed graft function, graft versus host disease, autoimmune diseases, e.g. rheumatoid arthritis, systemic lupus erythematosus, hashimoto's thyroidis, multiple sclerosis, myasthenia gravis, diabetes type I or II and the disorders associated therewith, vasculitis, pernicious anemia, Sjoegren syndrome, uveitis, psoriasis, Graves opthalmopathy, alopecia areata and others, allergic diseases, e.g. allergic asthma, atopic dermatitis, allergic rhinitis/conjunctivitis, allergic contact dermatitis, inflammatory diseases optionally with underlying aberrant reactions, e.g. inflammatory bowel disease, Crohn's disease or ulcerative colitis, intrinsic asthma, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, keratoconjunctivitis, myocarditis or hepatitis, e.g. acute or chronic hepatitis, ischemia/reperfusion injury, e.g. myocardial infarction, stroke, gut ischemia, renal failure or hemorrhage shock, traumatic shock, cancer, e.g. breast cancer, T cell lymphomas or T cell leukemias, nephrotic syndrome, infectious diseases, e.g. toxic shock (e.g. superantigen induced), septic shock, adult respiratory distress syndrome or viral infections, e.g. AIDS, viral hepatitis, e.g. hepatitis B or C, chronic bacterial infection, or neurodegenerative diseases, e.g. Alzheimer disease, amyotrophic lateral sclerosis or senile dementia. Examples of cell, tissue or solid organ transplants include e.g. pancreatic islets, stem cells, bone marrow, corneal tissue, neuronal tissue, heart, lung, combined heart-lung, kidney, liver, bowel, pancreas, trachea or oesophagus. For the above uses the required dosage will of course vary depending on the mode of administration, the particular condition to be treated and the effect desired.

In general, satisfactory results are indicated to be obtained systemically at daily dosages of from about 0.03 to 5.0 mg/kg per body weight. An indicated daily dosage in the larger mammal, e.g. humans, is in the range from about 0.5 mg to about 500 mg, conveniently administered, for example, in divided doses up to four times a day or in retard form. Suitable unit dosage forms for oral administration comprise from ca. 0.1 to 50 mg active ingredient.

The compounds of formula I may be administered by any conventional route, in particular enterally, e.g. orally, e.g. in the form of tablets or capsules, or parenterally, e.g. in the form of injectable solutions or suspensions, topically, e.g. in the form of lotions, gels, ointments or creams, or in a nasal or a suppository form. Pharmaceutical compositions comprising a compound of formula I in free form or in pharmaceutically acceptable salt form in association with at least one pharmaceutical acceptable carrier or diluent may be manufactured in conventional manner by mixing with a pharmaceutically acceptable carrier or diluent.

The compounds of formula I may be administered in free form or in pharmaceutically acceptable salt form e.g. as indicated above. Such salts may be prepared in conventional manner and exhibit the same order of activity as the free compounds.

In accordance with the foregoing the present invention further provides:
1.1 A method for preventing or treating disorders or diseases mediated by lymphocytes, e.g. such as indicated above, in a subject in need of such treatment, which method comprises administering to said subject an effective amount of a compound of formula I or a pharmaceutically acceptable salt thereof;
1.2 A method for preventing or treating acute or chronic transplant rejection or T-cell mediated inflammatory or autoimmune diseases, e.g. as indicated above, in a subject in need of such treatment, which method comprises administering to said subject an effective amount of a compound of formula I or a pharmaceutically acceptable salt thereof;
2. A compound of formula I, in free form or in a pharmaceutically acceptable salt form for use as a pharmaceutical, e.g. in any of the methods as indicated under 1.1 or 1.2 above.
3. A pharmaceutical composition, e.g. for use in any of the methods as in 1.1 or 1.2 above comprising a compound of formula I in free form or pharmaceutically acceptable salt form in association with a pharmaceutically acceptable diluent or carrier therefore.
4. A compound of formula I or a pharmaceutically acceptable salt thereof for use in the preparation of a pharmaceutical composition for use in any of the method as in 1.1 or 1.2 above.

The compounds of formula I may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g. a malignant cell anti-proliferative agent. For example, the compounds of formula I may be used in combination with a calcineurin inhibitor, e.g. cyclosporin A or FK 506; a mTOR inhibitor, e.g. rapamycin, 40-O-(2-hydroxyethyl)-rapamycin, CCI779, ABT578, AP23573, biolimus-7 or biolimus-9; an ascomycin having immuno-suppressive properties, e.g. ABT-281, ASM981, etc.; corticosteroids; cyclophosphamide; azathioprene; methotrexate; leflunomide; mizoribine; mycophenolic acid or salt; mycophenolate mofetil; 15-deoxyspergualine or an immunosuppressive homologue, analogue or derivative thereof; a PKC inhibitor, e.g. as disclosed in WO 02/38561 or WO 03/82859, e.g. the compound of Example 56 or 70; a JAK3 kinase inhibitor, e.g. N-benzyl-3,4-dihydroxy-benzylidene-cyanoacetamide α-cyano-(3,4-dihydroxy)-]N-benzylcinnamamide (Tyrphostin AG 490), prodigiosin 25-C (PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO 04/052359 or WO 05/066156; immunosuppressive monoclonal antibodies, e.g., monoclonal antibodies to leukocyte receptors, e.g., MHC, CD2, CD3, CD4, CD7, CD8, CD25, CD28, CD40, CD45, CD52, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent, e.g. paclitaxel, gemcitabine, cisplatinum, doxorubicin or 5-fluorouracil; or an anti-infectious agent.

Where the compounds of formula I are administered in conjunction with other immunosuppressive/immunomodulatory, anti-inflammatory, chemotherapeutic or anti-infectious therapy, dosages of the co-administered immunosuppressant, immunomodulatory, anti-inflammatory, chemotherapeutic or anti-infectious compound will of course vary depending on the type of co-drug employed, e.g. whether it is a steroid or a calcineurin inhibitor, on the specific drug employed, on the condition being treated and so forth. In accordance with the foregoing the present invention provides in a yet further aspect:
5. A method as defined above comprising co-administration, e.g. concomitantly or in sequence, of a therapeutically effective non-toxic amount of a compound of formula I and at least a second drug substance, e.g. an immunosuppressant, immunomodulatory, anti-inflammatory or chemotherapeutic drug, e.g. as indicated above.
6. A pharmaceutical combination, e.g. a kit, comprising a) a first agent which is a compound of formula I as disclosed herein, in free form or in pharmaceutically acceptable salt form, and b) at least one co-agent, e.g. an immunosuppressant, immunomodulatory, anti-inflammatory, chemotherapeutic or anti-infectious agent. The kit may comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula I and a co-agent, are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

1. A compound of formula I 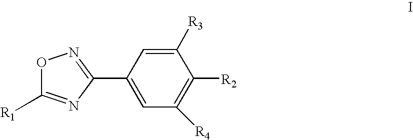
wherein
either
i. R₁ is a residue of formula (a) 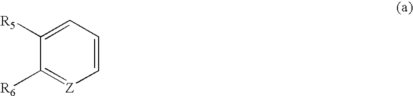
wherein Z is CH or N;
R₅ and R₆ are independently selected from the group consisting of H; halogen; C₁₋₈alkyl optionally substituted by OH, C₁₋₄alkoxy, ═N—OH or ═N—OC₁₋₄alkyl; halo-C₁₋₈alkyl; CN; C₁₋₈alkoxy; halo-C₁₋₈alkoxy; C₁₋₄alkylthio; and Rₐ—CO, wherein Rₐ is C₁₋₄alkyl, C₃₋₆cycloalkyl, phenyl, or phenyl-C₁₋₄alkyl; or phenyl; or R₅ and R₈ form together with the 2 carbon atoms to which they are attached another ring, so that R¹ is a residue of formula (b), 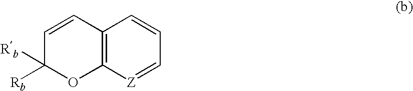
wherein Z is CH or N;
Rₐ is C₁₋₄alkyl; C₃₋₆cycloalkyl; phenyl; or phenyl-C₁₋₄alkyl; and
R_{b} and R′_{b} are independently C₁₋₄alkyl;
provided that at most one of R₅ and R₆ is H;
R₂ is —R₇—NR₈R₉ wherein R₇ is optionally substituted C₁₋₄alkylene; C₁₋₄alkylene wherein two bonds of a carbon atom of the alkyl chain form 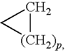
p being 1, 2 or 3; and R₈ and R₉ are independently selected from the group consisting of H, optionally substituted C₁₋₈alkyl, R_{d}—CO and a heterocyclic group, or R₈ and R₉ form together with the nitrogen atom to which they are bound an optionally substituted heterocyclic group; and
R_{d} is C₁₋₄alkyl; C₃₋₈cycloalkyl; phenyl; or phenyl-C₁₋₄alkyl; or
R₂ is an optionally substituted heterocyclic group; and
R₃ and R₄ are H;
provided that
1. when R₂ is —CH₂—NH₂, then R₁ is other than 2-CF₃-4-biphenylyl, 3-CF₃-4-trifluoroethoxy-phenyl or 3-CF₃-4-isopropoxy-phenyl; and
2. when R₂ is —C(CH₃)₂—NH₂, then R₁ is other than 2-CF₃-4-biphenyl)-4-biphenylyl; or
ii) R₁ is substituted biphenylyl, 4-phenoxy-phenyl or 4-(phenyl-C₁₋₄alkoxy)-phenyl wherein at least one of the phenyl groups is monosubstituted; or phenyl substituted by one or more substituents selected from the group consisting of halogen, nitrile, C₁₋₈alkyl, haloC₁₋₈alkyl, C₁₋₈alkoxy, haloC₁₋₈alkoxy, C₁₋₈alkoxy-C₁₋₈alkoxy, C₁₋₈alkyl-C₁₋₈alkoxy, C₁₋₈alkyl-haloC₁₋₈alkoxy, haloC₁₋₈alkyl-C₁₋₈alkoxy, haloC₁₋₈alkyl-haloC₁₋₈alkoxy, haloC₁₋₈alkoxy-C₁₋₈alkoxy, C₁₋₈alkoxy-haloC₁₋₈alkoxy, haloC₁₋₈alkoxy-haloC₁₋₈alkoxy, haloC₁₋₈-alkoxy-C₁₋₈alkyl, haloC₁₋₈alkoxy-haloC₁₋₈alkyl, C₂₋₆alkenyloxy, C₂₋₆alkynyloxy, C₃₋₆cycloalkyl, C₃₋₆cycloalkyl-C₁₋₄alkyl, C₃₋₆cycloalkyl-C₁₋₄alkoxy, C₃₋₆cycloalkyl-oxy, phenyl-C₁₋₄alkoxy and heterocyclic-C₁₋₄alkoxy;
R₂ is SO₂NH₂; or SO₂NH—R₁₀—COOH wherein R₁₀ is C₁₋₆alkylene optionally interrupted by O, S or C═CH₂;
one of R₃ and R₄ is H or C₁₋₄alkyl; and the other is C₁₋₄alkyl or haloC₁₋₄alkoxy;
provided that when R₂ is SO₂NH₂ and R₄ is ethyl, then R₁ is other than
a) biphenylyl substituted by CF₃ and fluoro; or
b) phenyl substituted by CF₃ and cyclohexyl;
or a salt thereof.

2. The compound according to claim 1, Wherein R₁ is a residue of formula (a),
wherein Z is CH or N;
R₅ and R₆ are independently selected from the group consisting of H; halogen; C₁₋₈alkyl optionally substituted by OH, C₁₋₄alkoxy, ═N—OH or ═N—OC₁₋₄alkyl; halo-C₁₋₈alkyl; CN; C₁₋₈alkoxy; halo-C₁₋₈alkoxy; C₁₋₈alkylthio; Rₐ—CO, wherein Rₐ is C₁₋₄alkyl, C₃₋₈cycloalkyl, phenyl, or phenyl-C₁₋₄alkyl; or phenyl;
provided that at most one of R₅ and R₆ is H.

3. The compound according to claim 1, wherein Z is CH.

4. The compound according to claim 1, wherein R₂ is R₇—NR₈R₉ or a heterocyclic ring attached by a carbon atom, wherein the variables have the meanings provided in claim 1.

5. The compound according to claim 1, wherein R₃ is H.

6. (canceled)

7. (canceled)

8. A pharmaceutical composition, comprising: the compound of formula I according to claim 1 or a pharmaceutically acceptable salt thereof, and a pharmaceutically acceptable diluent or carrier therefor.

9. (canceled)

10. A pharmaceutical combination, comprising: a) the compound of formula I according to claim 1, or a pharmaceutically acceptable salt thereof, and b) at least one co-agent selected from an immunosuppressant, immunomodulatory, anti-inflammatory, chemotherapeutic or anti-infectious agent.

11. (canceled)

12. A method of treatment of a disorder or disease mediated by lymphocytes, comprising: administering to a subject in need thereof, an effective amount of a compound according to claim 1 or a pharmaceutically acceptable salt thereof.

13. The method of claim 12 wherein the said disorder or disease is chronic transplant rejection, T-cell mediated inflammatory or autoimmune diseases.
